(19) United States
(12) Patent Application Publication
(10) Pub. No.: US 2014/0275826 A1
(43) Pub. Date: Sep. 18, 2014

(54) PHOTOACOUSTIC SENSORS FOR PATIENT MONITORING

(71) Applicant: Covidien LP, Mansfield, MA (US)

(72) Inventors: Youzhi Li, Longmont, CO (US); Qiaojian Huang, Broomfield, CO (US); Charles Keith Haisley, Boulder, CO (US); Sarah Lynne Hayman, Boulder, CO (US)

(21) Appl. No.: 13/842,466

(22) Filed: Mar. 15, 2013

(51) Int. Cl. A61B 5/00; A61B 5/02; A61B 5/0205; A61B 5/145; A61B 5/029

(52) U.S. Cl. CPC A61B 5/0095; A61B 5/7278; A61B 5/14546; A61B 5/14542; A61B 5/029; A61B 5/0205; A61B 5/02007
USPC 600/301; 600/407

(57) ABSTRACT

Various methods and systems for photoacoustic patient monitoring are provided. A photoacoustic system includes a light emitting component that emits one or more wavelengths of light into an interrogation region of a patient and an acoustic detector that detects acoustic energy generated by the interrogation region of the patient in response to the emitted light. A reflective coating is disposed on the light emitting component, the acoustic detector, the patient, or a combination thereof to direct the emitted light toward the interrogation region of the patient.

### BACKGROUND

The present disclosure relates generally to medical devices and, more particularly, to the use of photoacoustic spectroscopy in patient monitoring.

This section is intended to introduce the reader to various aspects of art that may be related to various aspects of the present disclosure, which are described and/or claimed below. This discussion is believed to be helpful in providing the reader with background information to facilitate a better understanding of the various aspects of the present disclosure. Accordingly, it should be understood that these statements are to be read in this light, and not as admissions of prior art.

In the field of medicine, medical practitioners often desire to monitor certain physiological characteristics of their patients. Accordingly, a wide variety of devices have been developed for monitoring patient characteristics. Such devices provide doctors and other healthcare personnel with the information they need to provide healthcare for their patients. As a result, such monitoring devices have become an indispensable part of modern medicine. Further, in certain medical contexts, it may be desirable to ascertain various localized physiological parameters, such as parameters related to individual blood vessels or other discrete components of the vascular system. Examples of such parameters may include oxygen saturation, hemoglobin concentration, perfusion, and so forth, for an individual blood vessel.

In one approach, measurement of such localized parameters is achieved via photoacoustic (PA) spectroscopy. PA spectroscopy utilizes light directed into a patient's tissue to generate acoustic waves that may be detected and resolved to determine localized physiological information of interest. In particular, the light energy directed into the tissue may be provided at particular wavelengths that correspond to the absorption profile of one or more blood or tissue constituents of interest. In some systems, the light is emitted as pulses (i.e., pulsed PA spectroscopy), though in other systems the light may be emitted in a continuous manner (i.e., continuous PA spectroscopy). The light absorbed by the constituent of interest results in a proportionate increase in the kinetic energy of the constituent (i.e., the constituent is heated), which results in the generation of acoustic waves. The acoustic waves may be detected and used to determine the amount of light absorption, and thus the quantity of the constituent of interest, in the illuminated region. For example, the detected ultrasound energy may be proportional to the optical absorption coefficient of the blood or tissue constituent and the fluence of light at the wavelength of interest at the localized region being interrogated (e.g., a specific blood vessel).

In many systems, the acoustic waves may be detected with an ultrasound transducer or transducer array. Unfortunately, the ultrasound transducer or array may also absorb light reflected and scattered off the skin tissue that is not indicative of the physiological information of interest, thus resulting in background signals at the transducer surface. These background signals may introduce noise into the obtained measurements, thus limiting the ability of a medical professional to determine the desired PA signal. Additionally, PA systems that utilize a high-intensity light emitter such as a laser introduce the risk of injury from the laser. Accordingly, there exists a need for PA systems and methods that safely obtain a desired PA signal.

### BRIEF DESCRIPTION OF THE DRAWINGS

Advantages of the disclosed techniques may become apparent upon reading the following detailed description and upon reference to the drawings in which:

FIG. 1 is a block diagram of a patient monitor and photoacoustic sensor in accordance with an embodiment;

FIG. 2 is a plot of an example photoacoustic signal illustrating a sensor-related photoacoustic signal effect;

FIG. 3 is a plot of an example photoacoustic signal generated with a reflective coated photoacoustic sensor in accordance with an embodiment;

FIG. 4 illustrates a photoacoustic sensor assembly having a reflective coating in accordance with an embodiment;

FIG. 5 illustrates a spacer component having a reflective coating disposed thereon in accordance with an embodiment;

FIG. 6 is a schematic illustrating an embodiment of a photoacoustic sensor having a reflective coating and being disposed on a patient;

FIG. 7 is a schematic illustrating another embodiment of a photoacoustic sensor having a reflective coating and being disposed on a patient;

FIG. 8 is a schematic illustrating an embodiment of a transmission type of photoacoustic sensor;

FIG. 9 is a schematic illustrating a reflective coating having a reflective material and an adhesive in accordance with an embodiment;

FIG. 10 is a schematic illustrating an embodiment of a photoacoustic system having an emitter and a detector positioned at a distance from one another on a patient;

FIG. 11 is a schematic illustrating a band-style photoacoustic sensor in accordance with an embodiment;

FIG. 12 is a schematic illustrating an ear clip style photoacoustic sensor in accordance with an embodiment;

FIG. 13 is a schematic illustrating an embodiment of a light delivery system having fiber coupling in a stacked arrangement;

FIG. 14 is a schematic illustrating another embodiment of a light delivery system having fiber coupling in a stacked arrangement;

FIG. 15 is a schematic illustrating another embodiment of a light delivery system having fiber coupling in an angled arrangement with a laser diode;

FIG. 16 is a schematic illustrating another embodiment of a light delivery system having angled fiber coupling;

FIG. 17 is a schematic illustrating an embodiment of a free space light delivery system;

FIG. 18 is a schematic illustrating another embodiment of a free space light delivery system;

FIG. 19 is a schematic illustrating another embodiment of a light delivery system having an optical fiber disposed in a prism;

FIG. 20 is a schematic illustrating an alternate embodiment of a light delivery system; and

FIG. 21 is a schematic illustrating another embodiment of a light delivery system.

### DETAILED DESCRIPTION OF SPECIFIC EMBODIMENTS

One or more specific embodiments of the present techniques will be described below. In an effort to provide a concise description of these embodiments, not all features of an actual implementation are described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

As described in detail below, presently disclosed embodiments of PA sensors, systems, and methods are provided for the measurement of various localized physiological parameters, such as parameters related to individual blood vessels or other discrete components of the vascular system. Examples of such parameters may include but are not limited to oxygen saturation, hemoglobin concentration, perfusion, cardiac output, and so forth, for an individual blood vessel. Certain features of the disclosed embodiments may reduce or eliminate the likelihood of generation of background signals present at the surface of the PA sensor, thus improving the likelihood that a blood PA signal in a vessel will be distinguishable in the acquired measurement.

In certain embodiments, the disclosed PA sensors may be utilized as part of a PA spectroscopy system in which light is directed into a patient's tissue to generate acoustic waves that may be detected and resolved to determine the localized physiological information of interest. In these embodiments, the light energy directed into the tissue is provided at particular wavelengths that correspond to the absorption profile of one or more blood or tissue constituents of interest. Disclosed embodiments may be utilized in PA spectroscopy systems in which the light is emitted as pulses (i.e., pulsed photoacoustic spectroscopy), as well as in systems in which the light is emitted in a continuous manner (i.e., continuous photoacoustic spectroscopy). One problem that may arise in photoacoustic spectroscopy may be attributed to the tendency of the emitted light to diffuse or scatter in the tissue of the patient. As a result, light emitted toward an internal structure or region, such as a blood vessel, may be diffused prior to reaching the region so that amount of light reaching the region is less than desired. Therefore, due to the diffusion of the light, less light may be available to be absorbed by the constituent of interest in the target region, thus reducing the acoustic waves generated at the target region of interest, such as a blood vessel.

In disclosed embodiments, the acoustic waves may be detected with an ultrasound transducer or transducer array, which may be made, for example, of piezoelectric materials such as lead zirconate titanate (PZT), polyvinylidene fluoride (PVDF), and so forth. The ultrasound transducer or array may also absorb light reflected and scattered off the skin tissue that is not indicative of the physiological information of interest, thus resulting in background signals at the transducer surface. However, presently disclosed embodiments may reduce or eliminate the likelihood that the blood PA signal in a vessel is buried by a strong background PA signal generated from the transducer surface. For example, in certain embodiments, one or more surfaces of the PA sensor may be provided with a reflective coating positioned such that the percentage of the light emitted from the PA sensor that is transmitted into the patient's tissue is increased compared to PA sensors without a reflective coating. As described in more detail below, the foregoing feature may offer distinct advantages over non-coated PA sensors because the background signals present due to transducer surface signals arising from light reflected off the patient's skin may be reduced. Additionally, in certain embodiments, a PA sensor includes a light emitter, such as a laser diode, oriented at an angle greater than the critical angle such that the emitted light is totally internally reflected when the sensor is in surrounding air, to reduce the risk of injury from the emitted light when the sensor is away from a patient's skin.

With this understanding, FIG. 1 depicts a block diagram of a photoacoustic spectroscopy system 8 in accordance with embodiments of the present disclosure. The system 8 includes a photoacoustic spectroscopy sensor 10 and a monitor 12. During operation, the sensor 10 emits spatially modulated light at certain wavelengths into a patient's tissue and detects acoustic shock waves generated in response to the emitted light. The monitor 12 is capable of calculating physiological characteristics based on signals received from the sensor 10 that correspond to the detected acoustic shock waves. The monitor 12 may include a display 14 and/or a speaker 16, which may be used to convey information about the calculated physiological characteristics to a user. The sensor 10 may be communicatively coupled to the monitor 12 via a cable or, in some embodiments, via a wireless communication link.

In one embodiment, the sensor 10 may include a light source 18 and an acoustic detector 20, such as an ultrasound transducer. The present discussion generally describes the use of pulsed light sources to facilitate explanation. However, as noted above, it should be appreciated that the photoacoustic sensor 10 may also be adapted for use with continuous wave light sources in other embodiments. Further, in certain embodiments, the light source 18 may be associated with one or more optical fibers for conveying light from one or more light generating components to the tissue site.

The photoacoustic spectroscopy sensor 8 may include the light source 18 and the acoustic detector 20 that may be of any suitable type. For example, in one embodiment, the light source 18 may include one, two, or more light emitting components (such as light emitting diodes) 21 adapted to transmit light at one or more specified wavelengths. In certain embodiments, the emitter 21 may include a laser diode or a vertical cavity surface emitting laser (VCSEL). The laser diode may be a tunable laser, such that a single diode may be tuned to various wavelengths corresponding to a number of different absorbers of interest in the tissue and blood. That is, the light may be any suitable wavelength or wavelengths (such as a wavelength between about 500 nm to about 1000 nm or between about 600 nm to about 900 nm) that is absorbed by a constituent of interest in the blood or tissue. For example, wavelengths between about 500 nm to about 600 nm, corresponding with green visible light, may be absorbed by deoxyhemoglobin and oxyhemoglobin. In other embodiments, red wavelengths (e.g., about 600 nm to about 700 nm) and infrared or near infrared wavelengths (e.g., about 800 nm to about 1000 nm) may be used. In one embodiment, the selected wavelengths of light may penetrate into the tissue of the patient 24 up to approximately 1 cm to approximately 2 cm. In disclosed embodiments that include the emitter 21, it should be understood that the emitter 21 may be coupled to an optical fiber.

The emitted light may be intensity modulated at any suitable frequency, such as from 1 MHz to 10 MHz or more. In one embodiment, the emitter 21 may emit pulses of light at a suitable frequency where each pulse lasts 10 nanoseconds or less and has an associated energy of a 1 mJ or less, such as between 1 mJ to 1 mJ. In such an embodiment, the limited duration of the light pulses may prevent heating of the tissue while still emitting light of sufficient energy into the region of interest to generate the desired acoustic waves when absorbed by the constituent of interest.

In one embodiment, as discussed herein, the light emitted by the light source 18 may be efficiently directed in the tissue of the patient 24 via a reflective coating 22. The reflective coating 22 may be positioned on any suitable surface of the light source 18, the PA sensor 10, the patient 24, or a combination thereof, depending on implementation-specific considerations. In accordance with disclosed embodiments, however, placement of the reflective coating 22 is such that the light that would be reflected and scattered off the tissue of the patient 24 to generate background signals present at the PA sensor 10 is partially or completely blocked. This may reduce or eliminate the likelihood of detection of transducer surface signals that affect the measurement of the desired PA signals. This feature may offer advantages over systems that do not include the reflective coating 22 because in certain embodiments the blood PA signal of the vessel may be more easily identified. Examples of suitable placements of the reflective coating 22 are discussed in more detail below.

In one embodiment, the acoustic detector 20 may be one or more ultrasound transducers suitable for detecting ultrasound waves emanating from the tissue in response to the emitted light and for generating a respective optical or electrical signal in response to the ultrasound waves. For example, the acoustic detector 20 may be suitable for measuring the frequency and/or amplitude of the acoustic waves, the shape of the acoustic waves, and/or the time delay associated with the acoustic waves with respect to the light emission that generated the respective ultrasound waves. In one embodiment an acoustic detector 20 may be an ultrasound transducer employing piezoelectric or capacitive elements to generate an electrical signal in response to acoustic energy emanating from the tissue of the patient 24, i.e., the transducer converts the acoustic energy into an electrical signal.

In some embodiments, the system 10 may also include any number or combination of additional medical sensors 23 or sensing components for providing information related to patient parameters that may be used in conjunction with the PA spectroscopy sensor 10. For example, suitable sensors may include sensors for determining blood pressure, blood constituents, respiration rate, respiration effort, heart rate, patient temperature, cardiac output, and so forth. Such information may be used, for example, to determine if the patient 24 is in shock or has an infection.

In one embodiment, the photoacoustic sensor 10 may include a memory or other data encoding component, depicted in FIG. 1 as an encoder 26. For example, the encoder 26 may be a solid state memory, a resistor, or combination of resistors and/or memory components that may be read or decoded by the monitor 12, such as via reader/decoder 28, to provide the monitor 12 with information about the attached sensor 10. For example, the encoder 26 may encode information about the sensor 10 or its components (such as information about the light source 18 and/or the acoustic detector 20). Such encoded information may include information about the configuration or location of photoacoustic sensor 10, information about the type of lights source(s) 18 present on the sensor 10, information about the wavelengths, pulse frequencies, pulse durations, or pulse energies which the light source(s) 18 are capable of emitting, information about the nature of the acoustic detector 20, and so forth. This information may allow the monitor 12 to select appropriate algorithms and/or calibration coefficients for calculating the patient's physiological characteristics, such as the amount or concentration of a constituent of interest in a localized region, such as a blood vessel.

In one embodiment, signals from the acoustic detector 20 (and decoded data from the encoder 26, if present) may be transmitted to the monitor 12. The monitor 12 may include data processing circuitry (such as one or more processors 30, application specific integrated circuits (ASICS), or so forth) coupled to an internal bus 32. Also connected to the bus 32 may be a RAM memory 34, a speaker 16 and/or a display 14. In one embodiment, a time processing unit (TPU) 40 may provide timing control signals to light drive circuitry 42, which controls operation of the light source 18, such as to control when, for how long, and/or how frequently the light source 18 is activated, and if multiple light sources are used, the multiplexed timing for the different light sources.

TPU 40 may also control or contribute to operation of the acoustic detector 20 such that timing information for data acquired using the acoustic detector 20 may be obtained. Such timing information may be used in interpreting the shock wave data and/or in generating physiological information of interest from such acoustic data. For example, the timing of the acoustic data acquired using the acoustic detector 20 may be associated with the light emission profile of the light source 18 during data acquisition. Likewise, in one embodiment, data acquisition by the acoustic detector 20 may be gated, such as via a switching circuit 44, to account for differing aspects of light emission. For example, operation of the switching circuit 44 may allow for separate or discrete acquisition of data that corresponds to different respective wavelengths of light emitted at different times.

In one embodiment, the received signal from the acoustic detector 20 may be amplified (such as via amplifier 46), may be filtered (such as via filter 48), and/or may be digitized if initially analog (such as via an analog-to-digital converter 50). The digital data may be provided directly to the processor 30, may be stored in the RAM 34, and/or may be stored in a queued serial module (QSM) 52 prior to being downloaded to RAM 34 as QSM 52 fills up. In one embodiment, there may be separate, parallel paths for separate amplifiers, filters, and/or A/D converters provided for different respective light wavelengths or spectra used to generate the acoustic data. The data processing circuitry (such as processor 30) may derive one or more physiological characteristics based on data generated by the photoacoustic sensor 12. For example, based at least in part upon data received from the acoustic detector 20, the processor 30 may calculate the amount or concentration of a constituent of interest in a localized region of tissue or blood using various algorithms. In one embodiment, these algorithms may use coefficients, which may be empirically determined, that relate the detected acoustic waves generated in response to pulses of light at a particular wavelength or wavelengths to a given concentration or quantity of a constituent of interest within a localized region. Further, by providing the reflective coating 22, the calculation of the desired physiological parameter may be improved due to the reduced presence of background signals present at the surface of the detector 20.

In one embodiment, processor 30 may access and execute coded instructions from one or more storage components of the monitor 12, such as the RAM 34, the ROM 60, and/or the mass storage 62. For example, code encoding executable algorithms may be stored in a ROM 60 or mass storage device 62 (such as a magnetic or solid state hard drive or memory or an optical disk or memory) and accessed and operated according to processor 30 instructions. Such algorithms, when executed and provided with data from the sensor 10, may calculate a physiological characteristic as discussed herein (such as the concentration or amount of a constituent of interest). Once calculated, the physiological characteristic may be displayed on the display 14 for a caregiver to monitor or review.

With the foregoing system discussion in mind, light emitted by the light source 18 of the photoacoustic sensor 10 may be used to generate acoustic signals in proportion to the amount of an absorber (e.g., a constituent of interest, such as a saline indicator) in a targeted localized region. However, as noted above, the emitted light may, in certain systems, be reflected and scattered off the skin of the patient 24 and absorbed by the detector 20, thereby generating undesirable background signals at the transducer surface. This effect is better understood by considering the plots 64 and 66 illustrated in FIGS. 2 and 3, respectively.

For example, FIG. 2 illustrates a graph 64 that depicts an example of an in vivo time domain photoacoustic (TDPA) test performed on human skin in accordance with one embodiment. The graph 64 includes a PA amplitude axis 68 and a time axis 70. The graph 64 also includes a plot 72 showing the TDPA test with a patient's vessel in the field of view of the imaging system as well as a plot 74 showing the TDPA test without the patient's vessel in the field of view of the imaging system. As shown in FIG. 2, a spike 76 is present in the plot 74 and is followed by a slow oscillation of the signal. This spike 76 and oscillation are caused by absorption of light scattered and reflected off the tissue of the patient, leading to generation of a strong PA signal from the surface of the PA sensor 10. In this graph 64, the effect of the background transducer surface signals on the ability of a processor or medical practitioner to identify a desired blood PA signal is shown. For example, a blood PA signal 78 present at approximately 11 micro-seconds along the time axis 70 is hidden by the strong background signal present due to light absorption at the surface of the PA sensor 10. That is, as illustrated, the background signal present at the transducer surface may reduce or eliminate the likelihood that the blood PA signal 78 is detectable.

FIG. 3 illustrates an example of a graph 66 obtained in accordance with a presently disclosed embodiment using an imaging system having the reflective coating 22 positioned on the surface of the sensor 10. Specifically, the graph 66 includes a PA amplitude axis 80 and a time axis 82. The graph 66 also includes a plot 84 obtained with the patient's vessel in the field of view of the imaging system. As shown, a PA signal 86 arising from light absorption at the sensor surface is reduced compared to the PA signal 76 shown in FIG. 2. The foregoing feature may enable a blood PA signal 88 present at approximately 11 micro-seconds along the time axis 82 to be more easily detectable compared to the blood PA signal 78 of FIG. 2. That is, the reduction of the background signal 76 that occurs due to inclusion of the reflective coating 22 better enables detection of the clinically relevant signal 88. Additionally, it should be noted that by including the reflective coating 22 in the embodiment shown in FIG. 3, the slow oscillation that follows spike 76 in plot 74 may also be reduced (e.g., as shown in FIG. 3) or eliminated. Again, these features may better enable a clinician to detect the clinically relevant spike 88 corresponding to the blood PA signal.

To that end, certain embodiments of the disclosure include photoacoustic sensors 10 with surface features that direct light into a patient's tissue and reduce absorption by the acoustic detector 20 and other sensor structures. FIG. 4 illustrates an embodiment of a photoacoustic sensor assembly 90 including an optically transparent and ultrasound coupling spacer 92, an acoustic detector 94, an optical fiber 96, and a housing or holder 98. The optical fiber 96 is coupled to the emitter 21. In certain embodiments, the spacer 92 is a Rexolite prism. Rexolite is utilized as the spacer 92 in some embodiments because of its low ultrasound attenuation and its ability to be machined to the prism shape, which facilitates tuning of the direction of ultrasound propagation during operation of the PA Sensor 90. However, in other embodiments, any desired spacer 92 having any desired features may be utilized, not limited to Rexolite, depending on implementation-specific considerations. For instance, in some embodiments, the spacer 92 may be any material having a low ultrasound impedance (i.e., an ultrasound impedance approximately equal or close to the ultrasound impedance of the tissue of the patient). For example, in one embodiment, the ultrasound impedance of the spacer 92 may be approximately 1.5-1.6 MRayls. Additionally, the sensor assembly 90 may include any suitable acoustic detector 94.

During operation of the PA sensor assembly 90, the optical fiber 96 emits light into the patient 24, and the acoustic detector 20 detects PA signals that are generated by a heating and thermal expansion effect within the interrogation region of the patient 24, as well as any light that has been reflected or scattered off the tissue of the patient 24. Accordingly, as best seen in FIG. 5, which illustrates a bottom surface 93 of the spacer 92 of FIG. 4, includes a reflective coating 100 disposed thereon. The reflective coating 100 functions to reduce or eliminate the presence of background signals at the transducer surface by increasing the amount of light from the emitter 96 that is directed into the patient. This feature may better enable the detection of the blood PA signal 88 because the strong PA signal 76 generated from light present at the transducer surface may be reduced or eliminated.

In presently disclosed embodiments, the reflective coating 100 may include any quantity and/or variety of suitable reflective materials, including but not limited to aluminum, copper, silver, gold, zinc, or a combination thereof. Additionally, during manufacturing, the reflective coating 100 may be applied to the desired region(s) of the assembly via any suitable manufacturing process, including but not limited to spraying, sputtering, or otherwise placing the reflective coating 100 on the desired region. Further, in certain embodiments, the placement and properties of the reflective coating 100 are chosen such that during operation, the reflective coating 100 doesn't significantly impede ultrasound transmission. For example, the material and/or dimensions (e.g., material thickness, density, etc.) of the reflective coating 100 may be chosen to minimize the effect of ultrasound transmission in a given application.

Again, by providing the reflective coating 100 on the spacer 92, the amount of light available to contribute to background signals present at the surface of the sensor 94 may be reduced or eliminated compared to sensors not having the reflective coating 100. That is, the reflective coating 100 may enable a reduction or elimination of light reflected and/or scattered by the tissue of the patient and reaching the detection surface of the sensor 94, thus reducing background surface signals. This reduction in background surface signals may better enable detection of the desired PA signal originating from the area of interest within the patient.

It should be noted that the placement of the reflective coating 100 is not limited to that which is shown in FIG. 5. Indeed, it is presently contemplated that the reflective coating 100 may be disposed on the light emitting component, the acoustic detector 20, the patient, or some combination thereof such that it is configured to direct the emitted light toward the interrogation region of the patient 24. FIGS. 6-12 illustrate example placements of the reflective coating 100 with respect to a patient, a sensor, and detector in accordance with disclosed embodiments, but the illustrated placements are merely examples and are not meant to limit the possible placements and configurations of the reflective coating 100. Indeed, the reflective coating 100 may be placed at any desirable location within the imaging environment, depending on implementation-specific considerations.

FIG. 6 is a schematic 102 illustrating one possible placement of the reflective coating 100 about a channel 106 disposed between portions of the acoustic detector 20. In this embodiment, the optical fiber 96 or emitter 20 emits light represented by arrows 104 toward the patient 24. Once emitted, the light 104 is directed toward the patient 24 by the reflective coating 100 disposed along inner surfaces of the acoustic detector 20. The light then enters the patient 24 to interact with the interrogation region of the patient 24, thus generating PA signals that are detected by the acoustic detector 20. In this embodiment, the positioning of the reflective coating 100 about the channel 106 through which light travels to reach the patient 24 may enable a greater percentage of the emitted signals 104 to reach the patient 24 compared to systems not including the reflective coating 100. This feature may reduce or eliminate the background signals present at the surface of the acoustic detector 20 due to reflected or scattered light, thus better enabling the PA signal from the area of interest to be detected by the acoustic detector 20. In other implementations, a spacer 92 may be positioned between the emitting point of the optical fiber 96 and the patient 24 instead of a channel 106.

FIG. 7 is a schematic 108 illustrating an alternate embodiment of the PA sensor assembly 10 having an alternate placement of the reflective coating 100. In this embodiment, the reflective coating 100 is positioned along the acoustic detector 20 to define the channel 106 through which the light 104 emitted by the emitter 96 travels toward the patient 24 as well as on the patient 24. That is, in some embodiments, the reflective coating 100 may be partially or entirely positioned on or in direct contact with the tissue of the patient 24 to aid in the direction of the light 104 toward the interrogation region of the patient 24 and the reduction of background signals. Additionally, it should be noted that the optical fiber 96 may also be included in the embodiment of FIG. 7 if desired in the given implementation.

Additionally, it should be noted that certain embodiments of the PA sensor assemblies described herein may be utilized in conjunction with other types of sensors that monitor physiological patient parameters and/or provide additional signal inputs for PA signal processing. The embodiments disclosed herein may be used in conjunction with the techniques disclosed in U.S. application Ser. No. 13/836,531, entitled, “PHOTOACOUSTIC MONITORING TECHNIQUE WITH NOISE REDUCTION,” to Dongyel Kang et al., assigned to Covidien LP, and filed on Mar. 15, 2013, the disclosure of which is incorporated by reference in its entirety herein for all purposes. For instance, in the schematic 108 shown in FIG. 7, a light detector 23 is positioned adjacent to the reflective coating on the patient 24. In other embodiments, the additional sensor or light detector 23 may be part of a pulse oximetry sensor, an oxygen sensor, a carbon dioxide sensor, or any other medical sensor. As such, it should be noted that in some embodiments, the reflective coating 100 and the PA sensor assemblies may be used either alone or in combination with additional medical sensors. This may enable coupling of photoacoustic technology with other types of technology to enable the collection of multiple types of parameters relating to physiological characteristics of the patient 24.

While certain disclosed embodiments relate to reflectance-type sensor configurations, it should be noted that in certain embodiments, it may be desirable to position the optical fiber 96 or emitter 21 and the detector 20 on opposite sides of an interrogation region of the patient 24, for example, to enable transmission type sensing. In such embodiments, the reflective coating 100 may be positioned on the patient 24 or on any portion of the sensor or detector suitable for directing light into the patient 24. For example, FIG. 8 illustrates an embodiment of a PA sensor assembly 110 having a body 112 with surfaces 114 and 116 that are configured to contact opposite sides of an area of interest of the patient 24 during operation. For instance, in one embodiment, an extremity of the patient 24 may be positioned between surfaces 114 and 116 of the PA sensor assembly 110, and a light emitting component disposed in the body 112 may emit one or more wavelengths of light into the extremity of the patient 24.

An acoustic detector, for example disposed in body 112 and under surface 116, may then detect acoustic energy generated by interrogating the patient with light emitted by an emitter positioned under surface 114. In this embodiment, the reflective coating 100 may be located at any desired location within the imaging environment or on the PA sensor assembly 110. For example, the reflective coating 100 may be disposed on the patient 24 as indicated by arrow 118, on the surface 114 as indicated by arrow 120, and/or on the surface 116 as indicated by arrow 122. Regardless of the position chosen in the given implementation, however, the reflective coating 100 is configured such that the emitted light is directed toward the interrogation region of the patient 24 and the presence of background signals at the surface of the sensor is reduced or eliminated.

As previously noted, in certain embodiments, the reflective coating 100 may be partially or entirely positioned on the patient 24 during operation. It should be further noted that in some embodiments, the reflective coating 100 may be positioned on the patient 24 independent of the sensor assembly 10. For example, as shown in the schematic of FIG. 9, the reflective coating 100 and the sensor assembly 10 may be independently placed on the patient 24, but are configured to cooperate during operation to direct light into an interrogation region of the patient 24. That is, the reflective coating 100 may be packaged or otherwise provided separately (e.g., as a sticker or painted-on material) from the sensor assembly 10, but may still be part of the functional sensor assembly when positioned on the patient 24 for use.

As depicted in FIG. 9, the reflective coating 100 may include one or more components that endow the coating 100 with reflective and/or other desired properties, such as adhesive properties, biocompatibility, disposability, and so forth. In the illustrated embodiment, the reflective coating 100 includes reflective material 124 and an adhesive 126. The reflective material 124 may include any reflecting component, such as but not limited to aluminum, copper, silver, zinc or a combination thereof. The adhesive 126 may be any suitable adhesive capable of facilitating the adherence of the reflective coating 100 to the tissue of the patient 24. Still further, it should be noted that in certain embodiments, the adhesive 126 may not be included in the reflective coating 100. For example, in one embodiment, the reflective material 124 may be painted or otherwise adhered to the patient 24 without use of the adhesive 126. It should be noted that in these embodiments, the reflective coating 100 may be configured as a removable and/or disposable device configured to be placed on the patient during operation and removed from the patient and discarded after operation.

Still further, in some embodiments, providing space between the emitting component and the detecting component may facilitate the generation and use of brighter light. For example, as shown in FIG. 10, it may be desirable to position the emitter 21 and the detector 20 at a distance 128 from one another on the patient 24, thus providing space between the emitter 21 and the detector 20 and enabling the light from the emitter 21 to more easily reach the patient 24. In this embodiment, the reflective coating 100 may be positioned at any desirable location within the imaging environment suitable for directing light toward the patient 24 without interfering with the desired transmission of the emitted light. For example, the reflective coating 100 may be placed on the emitter 21, on the acoustic detector 20, and/or on the patient 24. Indeed, in any given embodiment, the particular placement of the reflective coating 100 in the imaging environment may be chosen based on implementation specific considerations.

It should be noted that the PA sensor assembly 10 may be configured as any of a variety of suitable type of sensors designed for use on a region of interest of the patient 24. For example, FIG. 11 illustrates an embodiment of a band style sensor 130 having a band 132, an adhesive 134, and a sensor assembly 136 disposed thereon. In this embodiment, the reflective coating 100 may be placed, for example, on the adhesive 134 and/or on the sensor assembly 136. The bands may be configured, for example, to be placed around a patient's ear, neck, arm, leg, etc. such that the adhesive 134 and the sensor assembly 136 are positioned on a desired portion of the patient 24. In this way, the band 132 facilitates the proper placement of the sensor assembly 136, the adhesive 134, and the reflective coating 100 for operation. It should be noted, however, that in other embodiments the reflective coating 100 may be adhered or placed on the patient independent of the sensor assembly 136. For example, in one embodiment the reflective material 100 may be painted onto the patient's skin prior to the band 132 being positioned about the patient's body and the sensor assembly 136 being placed on the patient's region of interest.

FIG. 12 illustrates an alternate embodiment of a sensor assembly 138 including a band 140 and the PA sensor 10. In this embodiment, the sensor assembly 138 is configured as a clip-type sensor, for example, for clipping or resting on the patient ear of the patient 24. Here again, the reflective coating 100 may be included as part of the sensor 10 or it may be independently placed on the patient 24. Further, it should be noted that in this embodiment, as well as other embodiments described herein, the sensor 10 may be configured as a wireless sensor configured to communicate with monitor 12 via a wireless communication protocol. Indeed, presently disclosed embodiments are configured for use in both wired and wireless systems.

Additionally, it should be noted that use of the reflective coating 100 is consistent with a variety of types of light delivery systems. For example, the reflective coating 100 may be provided to reduce or prevent the generation of background signals in any of the light delivery systems illustrated in FIGS. 13-21, or any other light delivery system having any arrangement of system components. Specifically, FIG. 13 is a schematic 150 illustrating a light delivery system including a laser diode 152 that transmits light into an optical fiber 155 located in an optical channel 154. The optical fiber 155, guides the light and prevents the beam from dispersing, thus providing a higher light density beam when the beam reaches the surface of the patient's tissue. In another embodiment, the beam density of a fiber may be achieved by omitting the fiber and placing a reflective coating or foil along the surface of the optical channel 154.

The laser diode 152 may be connected by a cable 153 to a power source and/or medical device. As noted, in other implementations, the disclosed embodiments provided herein may also be configured as wireless sensors. In this arrangement, an ultrasound transducer 158, which functions as a detector during operation, is stacked with respect to a spacer 160 (which may be implemented as the same structure and/or materials as the spacer 92 of FIG. 4). During operation, the sensor assembly is positioned with respect to a patient such that a bottom surface 161 of the spacer 160 is in contact with a surface of the patient's tissue. Once the light is emitted and transmitted into the patient, the returning PA signal travels through the spacer 160 to the ultrasound transducer 158 where it is detected. It should be noted that the returning ultrasound signal follows this path in all the embodiments described below except the embodiments of FIGS. 17 and 19. This arrangement including the integral laser diode 152 may enable multiple sensor assembly sizes and configurations.

FIG. 14 is a schematic 162 illustrating a similar stacked arrangement that does not include the laser diode 152 and in which an optical fiber 155 extends through/between elements of the ultrasound transducer 158. Here again, the optical fiber 155 is illustrated within optical channel 154, but in other embodiments, the optical fiber 155 may be omitted and the optical channel 154 surface coated with a reflective coating or foil, depending on the implementation. In either of these embodiments, the reflective coating 100 may be positioned in any desired location in the imaging environment. Further, it should be noted that in some embodiments, the channel 154 may include some or all of the optical fiber 155. Additionally, in certain embodiments, the channel 154 may be partially or completely located in the spacer 160, while in other embodiments, the spacer 160 may be provided without the channel 154, which affects the light density as the beam will expand through the spacer 160.

In FIGS. 13 and 14, the angle at which light is delivered to the patient's tissue during operation is approximately 90 degrees (that is, substantially perpendicular). In some instances, during operation, this orthogonality may result in a reduced quantity of reflected light giving rise to background noise as compared to non-orthogonal designs and the higher light power density associated with these designs results in a stronger photoacoustic signal. Additionally, the embodiment of FIG. 13 may offer certain advantages, such as enabling control over a spot size of the emitted light that reaches the patient and accommodating a variety of sizes of ultrasound transducers 158.

FIG. 15 is a schematic 164 illustrating a light delivery system in which the spacer 160 includes an angled face or portion 166 that accommodates the angled laser diode 152 and an angled optical fiber 155 disposed within an angled optical channel 154. In some embodiments, by providing an angled light delivery system the likelihood that light will escape the spacer 160 when the light delivery device is removed from the surface of the patient's tissue (i.e., surface 161 is no longer in contact with the surface of the patient's tissue) may be significantly reduced or eliminated. This feature may offer advantages by decreasing the likelihood that emitted light reaches the patient or others in the surrounding environment when the assembly is not positioned for use on tissue (e.g., when the assembly is carried or lifted for repositioning by an operator). For example, when the light delivery device includes a high energy light source such as a laser diode, the angled optical channel can reduce the risk of eye injury, or other injury, due to the laser light not escaping the spacer 160 when the assembly is away from the patient's skin and/or surface 161 of the assembly is not in contact with the patient's tissue.

More specifically, an angle 165 between the optical fiber 155 and the side surface of the spacer 160 may be selected such that a light delivery angle is larger than the critical angle (i.e., the angle of incidence above which total internal reflection occurs) for the spacer 160 to air interface. For example, in embodiments in which Rexolite is used as the spacer 160, the angle 165 may be selected such that the light delivery angle is larger than approximately 39 degrees, which is the critical angle for the Rexolite to air interface. In embodiments in which the angle 165 is in this manner, the emitted light will be totally internally reflected when the sensor assembly is removed from the surface of the patient's tissue. Therefore, the emitted light will remain reflected within the sensor assembly, and will not emit into the surrounding environment, thereby reducing or eliminating the likelihood that an operator or others in the surrounding environment are exposed to the emitted light when the sensor assembly is removed from the patient. When the sensor assembly is in contact with the patient, the light delivery angle is less than the critical angle for the spacer 160 to tissue interface, and therefore the light is emitted into the patient tissue as desired for the PA response.

Similar to FIG. 15, FIG. 16 is a schematic 168 illustrating an angled light delivery system that does not include the laser diode 152, but instead, the optical fiber 155 extends through the optical channel 154 and outward from the spacer 160 through angled portion 166. Likewise, FIG. 18 is a schematic 174 illustrating another embodiment of an angled light delivery system having the laser diode 152 positioned on the angled portion 166 of the spacer 160. As discussed above, in embodiments in which the laser diode 152 (or other relatively high-powered light source 18) is used, the spacer 160 may provide additional patient safety by allowing the laser light to expand as it propagates through the spacer lowering the light density.

In the embodiments of FIGS. 15, 16, and 18, the PA signal is received on the bottom surface 161 of spacer 160 and transmitted through the spacer 160 to ultrasound transducer 158. Again, by selecting the angle 165 in such a way that the light delivery angle is larger than the critical angle for the spacer 160 to air interface, the likelihood that emitted light will be transmitted to the surrounding environment when the sensor assembly is removed from the patient's tissue may be reduced or eliminated.

Further, in the embodiments of FIGS. 15, 16, and 18, the reflective coating 100 may be positioned in any desired location in the imaging environment, such as on all or part of an interface between the ultrasound transducer 158 and the spacer 160. For example, the reflective coating 100 may include a reflective material positioned over the sensing face of the ultrasound transducer 158 to reflect light that reaches the ultrasound transducer 158 during operation. In certain embodiments, the reflective coating 100 may be thin with respect to the ultrasound transducer 158 and may have high light reflectivity in the infrared wavelength range.

Additionally, in certain embodiments (e.g., in FIGS. 13-16 and 19-21), the spacer 160 may or may not include the optical channel 154 along with the optical fiber 155. In particular, because the spacer 160 is optically transparent and light from the laser light source 18 (e.g., the laser diode 152 or the optical fiber 155) passes through without significant absorption, providing the spacer 160 without the channel 154 may be less complex from a manufacturing standpoint.

FIG. 17 is a schematic 170 illustrating an embodiment of an angled light delivery system having the laser diode 152 positioned on a ramp portion 172 of the spacer 160, which is configured in a prism shape. The PA signal is received by the spacer 160 on the bottom surface 161, and the PA signal travels vertically to ramp surface 172 from which it reflects orthogonally (due to the high mismatch of spacer to air interface) toward the ultrasound transducer 158. To maximize ultrasound detection, the ultrasound transducer 158 should be placed orthogonal to the PA signal reflected off ramp surface 172, which requires the angle 169 to be equal to angle 167. Further, as before, by selecting angle 167 in such a way that the light delivery angle is larger than the critical angle for the spacer 160 to air interface, the likelihood that emitted light will be transmitted to the surrounding environment when the sensor assembly is removed from the patient's tissue may be reduced or eliminated.

FIG. 19 is a schematic 180 illustrating an embodiment having the optical fiber 155 extending through the optical channel 154. In this embodiment, high light intensity may be achieved. Additionally, in this embodiment, as in the embodiments shown in FIGS. 14 and 16, due to the lower proximity the laser diode 152 to the ultrasound transducer 158, electrical crosstalk that may occur between the laser diode 152 and the ultrasound transducer 158, or parts thereof, may be eliminated. That is, in certain instances, placement of the ultrasound transducer 158 and the laser diode 152 in close proximity to one another during operation may give rise to crosstalk between their respective electrical cables or other parts of their respective assemblies, thus introducing noise into the signals carried by these cables. In embodiments where the laser diode 152 is not embedded in the PA sensor, this crosstalk may be reduced or eliminated.

Further, as described in more detail above with respect to FIG. 17, proper selection of angles 167 and 169 may enable total internal reflection to occur when the sensor assembly is removed from the surface of the patient's tissue. However, when the sensor assembly is placed on the surface of the patient, and after light is emitted into the patient's tissue, a PA signal is received by the spacer 160 on the bottom surface 161, and the PA signal travels vertically to ramp surface 172 from which it reflects orthogonally (due to the high mismatch of spacer to air interface) toward the ultrasound transducer 158.

FIG. 20 is a schematic 184 illustrating an alternate embodiment that may offer low sensor height profile advantages. In this embodiment, the ultrasound transducer 158 is positioned on the spacer 160 having a reduced thickness 186. The spacer 160 accommodates a portion of the optical fiber 155, which is located in an optical channel 154 directing the optical fiber 155 transversely through the spacer 160. The optical fiber 155 is coupled to a miniature prism 182, which turns the light toward the tissue interface surface of spacer 160.

FIG. 21 is a schematic 188 illustrating another light delivery system. In this embodiment, the spacer 160 is provided with a reduced thickness 189, which may be between approximately 0.5 mm to approximately 1 mm in some embodiments. Further, the optical fiber 155 extends through the ultrasound transducer 158, and a second optical fiber 190 and a prism 192 (e.g., a 1 mm prism) are provided to facilitate the introduction of light to optical fiber 155. During implementation, a fiber connector may be provided at the end of the second optical fiber 190. Additionally, in some embodiments, the diameter of the optical fiber 155 (e.g., approximately 2 mm) may be greater than the diameter of the second optical fiber 190 (e.g., approximately 1 mm), to increase efficiency of light transfer from optical fiber 190 to optical fiber 155.

In the designs shown in FIGS. 19, 20, and 21, certain advantages related to the emitted light spot size may be realized. For example, such designs may offer increased control over the light spot size. In some embodiments, this increased control over the light spot size may enable a greater tolerance when the operator is placing the sensor on the patient's tissue. That is, in these embodiments, the light spot size may be increased if desired to enable a larger possible placement area, for example, when probing a large vessel. Further, in some embodiments, the light spot size may be reduced in implementations in which an increased light power density is desired.

Additionally, in the embodiments of FIGS. 14, 16, and 19-21, electrical crosstalk present between the laser diode 152 and the ultrasound transducer 158 may be reduced or eliminated due to the positioning of the laser diode 152. That is, by omitting the laser diode 152 from the sensor portion of the assembly, electrical crosstalk may be reduced or eliminated.

Further, certain disclosed embodiments may accommodate use of cylindrical ultrasound transducers 158 having a variety of diameters (e.g., 5 mm, 7.5 mm, 10 mm, etc. For example, in the embodiments of FIGS. 13, 14, and 21, ultrasound transducers of multiple diameters may be accommodated due to the illustrated geometries that include the laser diode 152 and/or the optical fiber 155 centered with respect to the ultrasound transducer 158.

Additionally, it should be noted that the size of the ultrasound transducer 158 also has dependence on the PA signal strength and sensor placement tolerance. For example, the smaller the ultrasound transducer 158, the higher the PA signal strength and the smaller the sensor placement tolerance. The embodiments shown in FIGS.—13, 14, 20, and 21 may offer advantages by enabling ultrasound transducers 158 of any desired diameter to be utilized because the optical fiber 155 may be located approximately in the middle of the spacer 160.

Further, it should be noted that in one or more of the disclosed embodiments, the optical fiber 155 may terminate in a fiber connector (not shown in the illustrated embodiments) that facilitates coupling of the optical fiber 155 to other system components. Additionally, in some embodiments, the spacer 160 may be omitted, thus simplifying the manufacturing process.

While the disclosure may be susceptible to various modifications and alternative forms, specific embodiments have been shown by way of example in the drawings and have been described in detail herein. However, it should be understood that the embodiments provided herein are not intended to be limited to the particular forms disclosed. Rather, the various embodiments may cover all modifications, equivalents, and alternatives falling within the spirit and scope of the disclosure as defined by the following appended claims.

What is claimed is:

1. A photoacoustic system, comprising:
a light emitting component configured to emit one or more wavelengths of light into an interrogation region of a patient;
an acoustic detector configured to detect acoustic energy generated by the interrogation region of the patient in response to the emitted light; and
a reflective coating disposed on the light emitting component, the acoustic detector, the patient, or a combination thereof and configured to direct the emitted light toward the interrogation region of the patient.

2. The photoacoustic system of claim 1, wherein the reflective coating comprises aluminum, copper, silver, or a combination thereof.

3. The photoacoustic system of claim 1, comprising a monitor in communication with the light emitting component, the acoustic detector, or both.

4. The photoacoustic system of claim 1, wherein the light emitting component comprises one or more light emitting diodes, one or more laser diodes, a pulsed laser, a continuous wave laser, or a vertical cavity surface emitting laser.

5. The photoacoustic system of claim 1, wherein the acoustic detector comprises an ultrasound transducer.

6. The photoacoustic system of claim 1, comprising an optically transparent, low impedance spacer configured to be disposed adjacent to the acoustic detector and configured to be in contact with the interrogation region when the sensor is applied.

7. The photoacoustic system of claim 6, wherein the optically transparent, low impedance spacer is disposed between the acoustic detector and the interrogation region of the patient.

8. The photoacoustic system of claim 6, wherein the reflective coating is disposed on one or more faces of the optically transparent spacer.

9. The photoacoustic system of claim 6, wherein the optically transparent spacer comprises a translucent plastic produced by cross linking polystyrene with divinylbenzene.

10. A method, comprising:
emitting one or more wavelengths of light from a light source into an interrogation region of a patient;
directing the emitted light toward the interrogation region by providing reflective material along the path of the emitted light; and
detecting an acoustic response to the emitted light from the interrogation region of the patient with an acoustic detector.

11. The method of claim 10, comprising generating a signal corresponding to the detected acoustic response.

12. The method of claim 11, comprising processing the generated signal to determine a physiological parameter of the patient.

13. The method of claim 12, wherein the physiological parameter comprises total hemoglobin concentration, oxygen saturation, cardiac output, vessel specific oxygen saturation, or a combination thereof.

14. The method of claim 10, comprising emitting the one or more wavelengths of light as pulses.

15. The method of claim 10, comprising providing the reflective material as a coating disposed on an optically transparent spacer disposed at an interface between the acoustic detector and the interrogation region of the patient.

16. The method of claim 10, comprising providing the reflective material as a reflective assembly comprising a reflective coating and an adhesive configured to adhere to the interrogation region of the patient.

17. A photoacoustic system, comprising:
a sensor, comprising:
a body;
one or more light emitting components disposed in the body and configured to emit one or more wavelengths of light into an interrogation region of a patient;
one or more acoustic detectors disposed in the body and configured to detect acoustic energy generated by the interrogation region of the patient in response to the emitted light; and
a reflective material disposed on the body, the one or more light emitting components, the one or more acoustic detectors, the patient, or a combination thereof and configured to direct the emitted light toward the interrogation region of the patient; and
a patient monitor communicatively coupled to the sensor and configured to receive a signal from the one or more acoustic detectors that corresponds to the detected acoustic energy.

18. The photoacoustic system of claim 17, wherein the body comprises a first portion housing the light emitting component and a second portion housing the one or more acoustic detectors, and wherein the first portion is configured to be positioned on a first side of the interrogation region of the patient and the second portion is configured to be positioned on a second side of the interrogation region of the patient.

19. The photoacoustic system of claim 17, comprising an optically transparent spacer configured to be between the one or more light emitting components and the patient when the sensor is applied.

20. The photoacoustic system of claim 19, wherein the optically transparent spacer is configured to be between the one or more acoustic detectors and the patient when the sensor is applied.

21. A photoacoustic sensor, comprising:
a light emitting component configured to emit one or more wavelengths of light into an interrogation region of a patient;
an acoustic detector configured to detect acoustic energy generated by the interrogation region of the patient in response to the emitted light; and
an interface through which the light is configured to pass into the interrogation region of the patient,
wherein the light emitting component is oriented such that the light is emitted at an angle that is larger than the critical angle for the interface when the interface is opposed by air.

22. The photoacoustic sensor of claim 21, wherein the light emitting component is oriented by a spacer positioned between the light emitting component and the interrogation region of the patient.

23. The photoacoustic sensor of claim 21, wherein the orientation of the light emitting component provides substantially total internal reflection when the interface is opposed by air, and provides transmission of the light when the interface is opposed by the patient.
